(19) United States
(12) Patent Application Publication
(10) Pub. No.: US 2022/0193070 A1
(43) Pub. Date: Jun. 23, 2022

(54) METHODS FOR TREATING ABSENCE SEIZURES

(71) Applicants: XENON PHARMACEUTICALS INC., Burnaby (CA); THE UNIVERSITY OF BRITISH COLUMBIA, Vancouver (CA)

(72) Inventors: Robin Paul SHERRINGTON, North Vancouver (CA); Mary B. CONNOLLY, Vancouver (CA)

(21) Appl. No.: 17/532,909

(22) Filed: Nov. 22, 2021

Related U.S. Application Data
(60) Provisional application No. 63/116,881, filed on Nov. 22, 2020.

(51) Int. Cl. A61K 31/495; A61P 25/08

(52) U.S. Cl. CPC A61K 31/495; A61P 25/08

(57) ABSTRACT

In certain embodiments, the present disclosure is directed to methods and uses of flunarizine, including for treating absence seizures in a human patient, such as absence seizures in childhood absence epilepsy (CAE), wherein the methods comprise administering (e.g., orally) to a patient in need thereof a therapeutically effective amount of flunarizine. The present disclosure is further directed to various improved methods of therapy and administration of flunarizine.

### 1. BACKGROUND

Absence seizures occur in several epilepsy syndromes, most commonly in childhood absence epilepsy (CAE) and juvenile absence epilepsy (JAE). Absence seizures are brief and characterized by sudden onset and termination, impairment of awareness, and generalized spike-and-slow wave discharges (SWD) at a frequency of 2-6 per second (2-6 Hz) on electroencephalogram (EEG). The absence seizures associated with these syndromes have a slight variation across SWD frequency: CAE is characterized by 3 Hz SWD, the juvenile syndromes by 3-6 Hz SWD, and Lennox-Gastaut Syndrome (LGS) by 2-2.5 Hz SWD. In LSG, the slower frequency SWDs combined with the patient having the characteristic developmental delay have merited absence seizures in this syndrome being termed “atypical.” Oscillatory burst firing in the corticothalamic circuit is considered the underlying mechanism of absence seizures. The SWD on EEG associated with absence seizures constitutes a neurophysiologic deficit that is specific for absence epilepsy.

In general, ethosuximide, valproic acid, and lamotrigine are considered first line therapy for absence seizures. In a double blind, randomized clinical trial of ethosuximide, valproic acid, and lamotrigine in 446 subjects with CAE, >40% had inadequate response to treatment (Glauser et al., Epilepsia 2013, 54(1):141).

There is a great need for additional medications to treat refractory absence seizures. The present disclosure addresses this need by providing compositions and methods and uses for treating absence seizures, including absence seizures in CAE, and offers other related advantages.

### 2. SUMMARY

The present disclosure describes certain methods and uses for the small molecule flunarizine 1-[bis(4-fluorophenyl)methyl]-4-cinnamyl-piperazine). Flunarizine is indicated for the prophylaxis of migraine and the treatment of vertigo. The present disclosure provides methods and uses of flunarizine to treat absence seizures, for example in children, and compositions comprising flunarizine for carrying out the same.

In one embodiment, the present disclosure is directed to a method for treating absence epilepsy or absence seizures in a human patient, comprising administering (e.g., orally) to a patient in need thereof a therapeutically effective amount of flunarizine, thereby treating the absence epilepsy or absence seizures. In certain embodiments, the condition treated is childhood absence epilepsy (CAE) or juvenile absence epilepsy (JAE). In certain instances, the patient treated by the present methods and uses has exhibited treatment resistance to one or more epilepsy treatments, particularly two or more epilepsy treatments. Administering the therapeutically effective amount of flunarizine to the patient may result in a reduction in the number of absence seizures experienced by the patient in comparison to a baseline, a reduction of the duration of absence seizures experienced by the patient in comparison to a baseline, or both. Administering the therapeutically effective amount of flunarizine to the patient may result in correction of the patient's EEG pattern in whole or in part, by reducing the number or duration of episodes of SWD in comparison to a baseline.

In one embodiment, the present disclosure is directed to a method for treating absence epilepsy or absence seizures in a human patient, comprising administering (e.g., orally) to a patient in need thereof a therapeutically effective amount of flunarizine, wherein the patient has exhibited treatment resistance to one or more epilepsy treatments, particularly two or more epilepsy treatments.

In another embodiment, the present disclosure is directed to a method for treating absence epilepsy or absence seizures in a human patient, comprising identifying a patient that has exhibited treatment resistance to one or more epilepsy treatments, particularly two or more epilepsy treatments, and administering (e.g., orally) a therapeutically effective amount of flunarizine to the patient.

In one embodiment, the present disclosure is directed to a method for treating absence epilepsy or absence seizures in a human patient, comprising administering (e.g., orally) to a patient in need thereof a therapeutically effective amount of flunarizine as a second-line or later treatment for the absence epilepsy or absence seizures, particularly as a third-line or later treatment for the absence epilepsy or absence seizures.

In some aspects, the patient exhibits CAE. In certain aspects, the patient exhibits JAE. In other aspects, the patient does not exhibit Jeavons syndrome. In yet other aspects, the patient does not exhibit Lennox-Gastaut Syndrome. In certain embodiments, the patient does not exhibit another epilepsy disease, disorder, or condition other than CAE or JAE.

In certain instances, the patient is less than 21 years of age, the patient is from 3 to 20 years of age, or the patient is from 4 to 18 years of age.

In some aspects, the patient exhibits absence seizures with hyperventilation, the patient exhibits photosensitive epilepsy (such as sunflower syndrome), or the patient also exhibits tonic-clonic seizures. In other aspects, the patient exhibits generalized polyspike or spike and wave discharges at 2 to 6 Hz when measured by electroencephalogram. In certain instances, the patient exhibits generalized spike and wave discharges at 3 Hz when measured by electroencephalogram.

In some instances, the patient treated by the present methods and uses has exhibited treatment resistance to two or more epilepsy treatments. In other instances, the patient has exhibited treatment resistance to one or more of topiramate, zonisamide, levetiracetam, a benzodiazepine (such as clobazam or clonazepam), stiripentol, amantadine, ethosuximide, valproic acid or valproate, divalproex sodium, or lamotrigine. In certain aspects, the patient has exhibited treatment resistance to one or more of ethosuximide, valproic acid or valproate (e.g., sodium valproate), or lamotrigine.

In some instances, the patient treated by the present methods and uses is concurrently being treated with one or more additional epilepsy treatments. In other instances, the patient is concurrently being treated with two or more additional epilepsy treatments. In some aspects, the patient is concurrently being treated with topiramate, zonisamide, levetiracetam, a benzodiazepine (such as clobazam or clonazepam), stiripentol, amantadine, ethosuximide, valproic acid or valproate, divalproex sodium, or lamotrigine. In certain instances, the patient is concurrently being treated with ethosuximide, valproic acid or valproate (e.g., sodium valproate), or lamotrigine.

In some aspects, the patient treated by the present methods and uses is not concurrently being treated with an additional epilepsy treatment. In certain embodiments, the patient is not concurrently being treated with one or more of topiramate, zonisamide, levetiracetam, a benzodiazepine (such as clobazam or clonazepam), stiripentol, amantadine, ethosuximide, valproic acid or valproate, divalproex sodium, or lamotrigine. In some instances, the patient is not concurrently being treated with one or more of ethosuximide, valproic acid or valproate (e.g., sodium valproate), or lamotrigine. In certain embodiments, the patient is not concurrently being treated with valproate (e.g., sodium valproate).

In other aspects, the patient is on a ketogenic diet. In some aspects, the patient is not on a ketogenic diet.

In some embodiments, in addition to absence seizures, the patient treated by the present methods and uses also exhibits one or more of atypical absence seizures, focal seizures (such as temporal lobe seizures, frontal lobe seizures, occipital lobe seizures, and parietal lobe seizures), generalized seizures (such as absence seizures, myoclonic seizures, generalized convulsive seizures, and grand mal seizures), symptomatic generalized epilepsy, progressive myoclonic epilepsy, reflex epilepsy, neonatal onset epilepsy syndromes or seizure disorders (such as Ohtahara syndrome and benign familial neonatal seizures), infantile onset epilepsy syndromes or seizure disorders (such as infantile spasms or West syndrome and Dravet syndrome), neurologic disorders (such as Rett syndrome, Angelman syndrome, tuberous sclerosis, and Sturge-Weber syndrome), childhood onset epilepsy syndromes or seizure disorders (such as febrile seizures, Landau-Kleffner syndrome, Lennox-Gastaut syndrome, Rasmussen syndrome, gelastic epilepsy, benign rolandic epilepsy, benign occipital epilepsy, and juvenile myoclonic epilepsy).

In certain instances, the patient treated by the present methods and uses is female. In other instances, the patient treated by the present methods and uses is male. In certain instances, the patient exhibits absence seizures with myoclonic seizures. In other instances, the patient exhibits absence seizures without myoclonic seizures. In yet other instances, the patient exhibits absence seizures without atypical absence seizures.

In other embodiments, the present methods comprise administering 0.5-50 mg/day of flunarizine to the patient, administering 1-25 mg/day of flunarizine to the patient, administering 2-15 mg/day of flunarizine to the patient, administering 5 mg/day of flunarizine to the patient, or administering 10 mg/day of flunarizine to the patient. In certain embodiments, the daily dose of flunarizine is administered in a single dose per day. In other embodiments, the daily dose of flunarizine is administered in as multiple doses per day, such as two doses per day.

These and other aspects of this disclosure will be apparent upon reference to the following detailed description. To this end, various references are set forth herein which describe in more detail certain background information and procedures and are each hereby incorporated by reference in their entirety.

### 3. BRIEF DESCRIPTION OF THE DRAWINGS

FIG. 1 shows a summary of the timeline of visits and treatment in the clinical protocol of Example 1.

### 4. DETAILED DESCRIPTION

The present disclosure relates to novel and improved methods and uses for flunarizine, particularly for treatment of childhood absence epilepsy (CAE) or juvenile absence epilepsy (JAE) by administering flunarizine to a human patient in need thereof, including by oral administration.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

4.1. Definitions

As used in the specification and appended claims, unless specified to the contrary, the following terms and abbreviations have the meaning indicated:

“Epilepsy treatments” as used herein refers to therapeutic applications associated with treating epilepsy or a disorder that causes epileptic seizures or one or more mechanisms underlying epilepsy or a disorder that causes epileptic seizures in a human patient.

“Flunarizine” as used herein refers to the compound having the following formula:

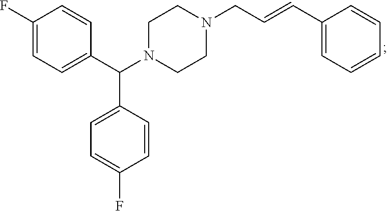 [SMILES: Fc1ccc(C(c2ccc(F)cc2)N2CCN(C/C=C/c3ccccc3)CC2)cc1]

and having a chemical name of 1-[bis(4-fluorophenyl)methyl]-4-cinnamyl-piperazine. Reference to “flunarizine” herein also includes pharmaceutically acceptable salts of flunarizine, unless stated otherwise. Flunarizine was marketed in 5 mg capsules as SIBELIUM® by Janssen Pharmaceutica Ltd. at least as early as 1987 for the prophylaxis of migraine and treatment of vertigo and is commercially available in free base or salt form from several chemical vendors, including Sigma-Aldrich of St. Louis, Mo.; Alfa Chemistry of Ronkonkoma, N.Y.; Pi Chemicals of Shanghai, China; and Yuhao Chemical of Hangzhou, China, among others. Flunarizine is used in the methods and uses described herein.

“Second-line or later treatment” as used herein refers to a treatment for the indicated disease, disorder, or condition (e.g., absence epilepsy, absence seizures, CAE, JAE, etc.) subsequent to one or more earlier treatments for the indicated disease, disorder, or condition that did not involve flunarizine and for which the human patient exhibited treatment resistance, i.e., the patient's disease, disorder, or condition was refractory to the one or more earlier treatments. Second-line or later treatment includes second-line treatment (i.e., a treatment that follows one earlier treatment, a “first-line treatment,” to which the patient exhibited treatment resistance), third-line treatment (i.e., a treatment that follows two earlier treatments to which the patient exhibited treatment resistance), fourth-line treatment (i.e., a treatment that follows three earlier treatments to which the patient exhibited treatment resistance), etc.

“Therapeutically effective amount” as used herein refers to an amount of flunarizine that is sufficient to treat the stated disease, disorder, or condition or have the desired stated effect on the disease, disorder, or condition or one or more mechanisms underlying the disease, disorder, or condition in a human subject. In certain embodiments, when flunarizine is administered for the treatment of absence seizures, such as absence seizures in childhood absence epilepsy (CAE), therapeutically effective amount refers an amount of flunarizine which, upon administration to a human, treats or ameliorates absence seizures in the human, or exhibits a detectable therapeutic effect in the absence seizures. The effect can be detected by, for example, a reduction in the number of absence seizures experienced by the patient in a given time period in comparison to a baseline, a reduction of the duration of absence seizures experienced by the patient in comparison to a baseline, or both. In certain embodiments, the number in a given time period or duration of absence seizures experienced by the patient is reduced by at least 50%, such by at least 60%, by at least 70%, by at least 80%, or by at least 90%. Alternatively, the effect can be detected by a reduction in the number of episodes of SWD in a given time period or duration of episodes of SWD in the patient's EEG in comparison with a baseline, or both. In certain embodiments, the number of episodes of SWD in a given time period or duration of episodes SWD in the patient's EEG is reduced by at least 50%, such as by at least 60%, by at least 70%, by at least 80%, or by at least 90%.

“Treatment” or “treating” as used herein refers to therapeutic applications associated with administering flunarizine that ameliorate the indicated disease, disorder, or condition or one or more underlying mechanisms of said disease, disorder, or condition, including slowing or stopping progression of the disease, disorder or condition or one or more of the underlying mechanisms in a human subject. In certain embodiments, when flunarizine is administered for the treatment of absence seizures, such as absence seizures in childhood absence epilepsy (CAE), treatment refers to therapeutic applications that reduce the frequency or duration of absence seizures. Treatment of absence seizures may also modulate neuronal cellular behavior toward a normal state that would be observed in the absence of absence seizures.

“Treatment resistance” as used herein with reference to an epilepsy treatment refers to the result of a human patient having not demonstrated an amelioration of an indicated epilepsy or disorder that causes epileptic seizures despite having been treated with the epilepsy treatment. In the context of reducing the frequency of absence seizures, treatment resistance refers to a human patient not having experienced a 20% or greater reduction in the frequency of absence seizures despite having been treated with the epilepsy treatment.

4.2. Embodiments

In some embodiments, the present disclosure is directed to methods or uses involving administration of flunarizine, including a method of treating absence seizures in a human patient, comprising administering (e.g., orally) to a patient in need thereof a therapeutically effective amount of flunarizine, thereby treating the absence seizures. In certain instances, the absence seizures occur in an epilepsy syndrome exhibited by the patient, such as childhood absence epilepsy (CAE), juvenile absence epilepsy (JAE), and juvenile myoclonic epilepsy (JME). In some embodiments, the method of treating absence seizures treats the CAE, JAE, or JME. In certain embodiments, the amount of flunarizine administered is sufficient to reduce the duration of absence seizures experienced by the patient, the frequency of absence seizures experienced by the patient, or both. In other embodiments, the amount of flunarizine administered is sufficient to reduce the number or duration of episodes of SWD in the patient's EEG. In particular embodiments, the epilepsy disorder treated by administration of flunarizine is childhood absence epilepsy (CAE).

In one embodiment, the present disclosure is directed to a method for treating childhood absence epilepsy (CAE) or juvenile absence epilepsy (JAE) in a human patient, comprising administering (e.g., orally) to a patient in need thereof a therapeutically effective amount of flunarizine, thereby treating the CAE or JAE. In particular embodiments, the present disclosure is directed to a method for treating CAE in a human patient, comprising administering (e.g., orally) to a patient in need thereof a therapeutically effective amount of flunarizine, thereby treating the CAE. In certain instances, the patient treated by the present methods and uses has exhibited treatment resistance to one or more epilepsy treatments.

In some embodiments, the present disclosure is directed to a method for treating childhood absence epilepsy (CAE) or juvenile absence epilepsy (JAE) in a human patient, comprising administering (e.g., orally) to a patient in need thereof a therapeutically effective amount of flunarizine as a first-line treatment, thereby treating the CAE or JAE. In certain such instances, the patient is not concurrently being treated with an additional epilepsy treatment.

In one embodiment, the present disclosure is directed to a method for treating absence epilepsy or absence seizures in a human patient, comprising administering (e.g., orally) to a patient in need thereof a therapeutically effective amount of flunarizine, wherein the patient has exhibited treatment resistance to one or more epilepsy treatments.

In another embodiment, the present disclosure is directed to a method for treating absence epilepsy or absence seizures in a human patient, comprising identifying a patient that has exhibited treatment resistance to one or more epilepsy treatments, and administering (e.g., orally) a therapeutically effective amount of flunarizine to the patient.

In one embodiment, the present disclosure is directed to a method for treating absence epilepsy or absence seizures in a human patient, comprising administering (e.g., orally) to a patient in need thereof a therapeutically effective amount of flunarizine as a second-line or later treatment for the absence epilepsy or absence seizures. In some embodiments, administering a therapeutically effective amount of flunarizine to the patient is a third-line treatment (or a third-line or later treatment) for the absence epilepsy or absence seizures. In other embodiments, administering a therapeutically effective amount of flunarizine to the patient is a fourth-line treatment (or a fourth-line or later treatment) for the absence epilepsy or absence seizures. In yet further embodiments, administering a therapeutically effective amount of flunarizine to the patient is a fifth-line treatment (or a fifth-line or later treatment) for the absence epilepsy or absence seizures.

Administering the therapeutically effective amount of flunarizine to the patient according to the above methods and uses may result in a reduction in the number of absence seizures experienced by the patient in comparison to a baseline, a reduction of the duration of absence seizures experienced by the patient in comparison to a baseline, or both. Administering the therapeutically effective amount of flunarizine to the patient according to the above methods and uses may result in correction of the patient's EEG pattern in whole or in part, by reducing the number or duration of episodes of SWD in comparison to a baseline.

In certain instances, the patient is less than 21 years of age. In some embodiments, the patient is from 3 to 20 years of age. In certain instances, the patient is from 4 to 18 years of age. In some instances, the patient is from 4 to 16 years of age.

In some aspects, prior to treatment, the patient exhibits absence seizures with hyperventilation. In certain embodiments, prior to treatment, the patient exhibits photosensitive epilepsy (such as sunflower syndrome). In some embodiments, prior to treatment, the patient exhibits tonic-clonic seizures. In other aspects, prior to treatment, the patient exhibits generalized polyspike or spike and wave discharges at 2 to 6 Hz when measured by electroencephalogram. In certain instances, prior to treatment, the patient exhibits generalized spike and wave discharges at 3 Hz when measured by electroencephalogram.

In certain instances, the patient treated by the present methods and uses has exhibited treatment resistance to one or more epilepsy treatments, such as resistance to two or more, three or more, four or more, or five or more epilepsy treatments. In some instances, those epilepsy treatments to which the patient has exhibited treatment resistance are selected from one or more of topiramate, zonisamide, levetiracetam, a benzodiazepine (such as clobazam or clonazepam), stiripentol, amantadine, ethosuximide, valproic acid or valproate, divalproex sodium, or lamotrigine. In certain aspects, the patient has exhibited treatment resistance to one or more of ethosuximide, valproic acid or valproate (e.g., sodium valproate), or lamotrigine.

In some instances, the patient treated by the present methods and uses is concurrently being treated with one or more additional epilepsy treatments. In other instances, the patient is concurrently being treated with two or more additional epilepsy treatments. In some aspects, the patient is concurrently being treated with one or more of topiramate, zonisamide, levetiracetam, a benzodiazepine (such as clobazam or clonazepam), stiripentol, amantadine, ethosuximide, valproic acid or valproate, divalproex sodium, or lamotrigine. In certain instances, the patient is concurrently being treated with one or more of ethosuximide, valproic acid or valproate (e.g., sodium valproate), or lamotrigine.

In some aspects, the patient treated by the present methods and uses is not concurrently being treated with an additional epilepsy treatment. In certain embodiments, the patient is not concurrently being treated with one or more of topiramate, zonisamide, levetiracetam, a benzodiazepine (such as clobazam or clonazepam), stiripentol, amantadine, ethosuximide, valproic acid or valproate, divalproex sodium, or lamotrigine. In some instances, the patient is not concurrently being treated with one or more of ethosuximide, valproic acid or valproate (e.g., sodium valproate), or lamotrigine. In certain embodiments, the patient is not concurrently being treated with valproate (e.g., sodium valproate).

In some instances, the patient is on a ketogenic diet. In other aspects, the patient is not on a ketogenic diet.

In some embodiments, in addition to absence seizures, the patient treated by the present methods and uses also exhibits one or more of atypical absence seizures, focal seizures (such as temporal lobe seizures, frontal lobe seizures, occipital lobe seizures, and parietal lobe seizures), generalized seizures (such as absence seizures, myoclonic seizures, generalized convulsive seizures, and grand mal seizures), symptomatic generalized epilepsy, progressive myoclonic epilepsy, reflex epilepsy, neonatal onset epilepsy syndromes or seizure disorders (such as Ohtahara syndrome and benign familial neonatal seizures), infantile onset epilepsy syndromes or seizure disorders (such as infantile spasms or West syndrome and Dravet syndrome), neurologic disorders (such as Rett syndrome, Angelman syndrome, tuberous sclerosis, and Sturge-Weber syndrome), childhood onset epilepsy syndromes or seizure disorders (such as febrile seizures, Landau-Kleffner syndrome, Lennox-Gastaut syndrome, Rasmussen syndrome, gelastic epilepsy, benign rolandic epilepsy, benign occipital epilepsy, and juvenile myoclonic epilepsy).

In some aspects, the patient exhibits CAE. In certain aspects, the patient exhibits JAE or JME. In other aspects, the patient does not exhibit Jeavons syndrome. In yet other aspects, the patient does not exhibit Lennox-Gastaut Syndrome. In certain embodiments, the patient does not exhibit another epilepsy disease, disorder, or condition other than CAE, JAE, or JME. In some embodiments, the patient docs not exhibit another epilepsy disease, disorder, or condition other than CAE.

In certain instances, the patient exhibits absence seizures with myoclonic seizures. In other instances, the patient exhibits absence seizures without myoclonic seizures. In yet other instances, the patient exhibits absence seizures without atypical absence seizures.

In certain instances, the patient treated by the present methods and uses is female. In other instances, the patient treated by the present methods and uses is male.

In one embodiment, the methods and uses described herein, such as the method of or use in treating absence seizures, such as absence seizures in childhood absence epilepsy (CAE) or juvenile absence epilepsy (JAE), in a human patient, is achieved by administering (e.g., orally) to a patient in need thereof a therapeutically effective amount of flunarizine, such as from about 0.02 mg/kg to about 2.0 mg/kg. More specific representative amounts include 0.01 mg/kg, 0.02 mg/kg, 0.05 mg/kg, 0.06 mg/kg, 0.08 mg/kg, 0.10 mg/kg, 0.12 mg/kg, 0.14 mg/kg, 0.15 mg/kg, 0.16 mg/kg, 0.18 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg and 2 mg/kg, or any range of amounts created by using two of the aforementioned amounts as endpoints. In some aspects, the method or use includes administering (e.g., orally) 0.02-0.5 mg/kg of flunarizine, such as 0.05-0.2 mg/kg of flunarizine. In some aspects, the method includes administering (e.g., orally) 0.1-0.7 mg/kg of flunarizine. In some aspects, the method includes administering (e.g., orally) 0.08 mg/kg of flunarizine +/−10%. In certain aspects, the method includes administering (e.g., orally) 0.16 mg/kg of flunarizine +/−10%.

In some embodiments, the methods and uses described herein, such as the method of or use in treating absence seizures, such as absence seizures in childhood absence epilepsy (CAE) or juvenile absence epilepsy (JAE), in a human patient, is achieved by administering (e.g., orally) to a patient in need thereof a therapeutically effective amount of flunarizine, such as 0.5 mg to 100 mg of flunarizine in a single or divided doses. For example, the method can include administering (e.g., orally), in a single or divided doses, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg or administering (e.g., orally) any range of amounts created by using two of the aforementioned amounts as endpoints. In some aspects, the method or use includes oral administration of 0.5 mg to 50 mg of flunarizine in a single or divided doses, such as 1 to 25 mg, 2 to 15 mg, or 5 to 10 mg of flunarizine in a single or divided doses. In some aspects, method or use includes the oral administration of a single or divided dose of 1, 2, 5, 6, 7, 8, 9, 10, 11, 12, 13, 14, 15, 20, or 25 mg of flunarizine, particularly 5 or 10 mg of flunarizine.

In some aspects, the methods and uses described herein, such as the method of or use in treating absence seizures, such as absence seizures in childhood absence epilepsy (CAE) or juvenile absence epilepsy (JAE), in a human patient, is achieved by administering (e.g., orally) to a patient in need thereof at least 5 mg of flunarizine, such as at least 10, 15, 20, 30, 40, or 50 mg of flunarizine. In some embodiments, the methods and uses described herein, such as the method of or use in treating a childhood absence epilepsy (CAE) in a human patient, is achieved by administering (e.g., orally) to a patient in need thereof at least 50 mg of flunarizine per day, such as at least 60, 70, 80, 90, or 100 mg of flunarizine per day.

In some embodiments, the methods and uses described herein, such as the method of or use in treating absence seizures, such as absence seizures in childhood absence epilepsy (CAE) or juvenile absence epilepsy (JAE), in a human patient, is achieved by administering (e.g., orally) to a patient in need thereof a therapeutically effective amount of flunarizine per day, such as 0.5 to 500 mg of flunarizine per day, such as 1 to 100 mg, 2 to 50 mg, or 2 to 25 mg of flunarizine per day. For example, the method or use can include administering (e.g., orally) about 1 mg, about 2 mg, about 3 mg, about 4 mg, about 5 mg, about 6 mg, about 8 mg, about 10 mg, about 12 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, or about 500 mg of flunarizine per day, or administering (e.g., orally) per day a range of amounts created by using two of the aforementioned amounts as endpoints. In some aspects, the method or use includes orally administering 1 to 100 mg of flunarizine per day, such as 1, 2, 3, 4, 5, 6, 7, 8, 9, 10, 11, 12, 13, 14, 15, 16, 17, 18, 19, 20, 25, 30, 35, or 40 mg to 75, or 100 mg of flunarizine per day, including 2 to 25 mg per day. In some aspects, the oral administration includes 2, 5, 8, 10, 12, 15, 20, or 25 mg of flunarizine per day, such as 5 or 10 mg per day. In some aspects, the method or use includes oral administration of a single or divided daily dose of 5 mg of flunarizine. In other aspects, the method or use includes oral administration of a single or divided daily dose of 10 mg of flunarizine. Suitable examples of divided daily doses include twice, three times, or four times a day, particularly twice a day.

In certain instances, the above daily doses of flunarizine are administered (e.g., orally) as multiple doses per day, such as in two, three, four, or five doses per day, particularly two doses per day. For example, a daily dose of 5 mg, maybe administered in two 2.5 mg doses during the day, such as in the morning and at bed. In another example, a daily dose of 10 mg, maybe administered in two 5 mg doses during the day, such as in the morning and at bed.

In some embodiments, the above daily doses of flunarizine are administered (e.g., orally) as a single dose. For example, 1 to 100 mg, 2 to 50 mg, or 2 to 25 mg of flunarizine per day can be orally administered as a single dose, including 2-15 mg and 5-10 mg per day as a single dose, such as 2-8 mg per day or 7-13 mg per day as a single dose. In some embodiments, the above daily doses of flunarizine are administered (e.g., orally) as a single dose in the morning or at night. In some embodiments, the above daily doses of flunarizine are administered (e.g., orally) as a divided dose, e.g. by administration of a first dose in the morning and a second dose at night. Relatedly, any of the doses of flunarizine discussed in the preceding paragraphs may be included in a unit dosage form.

In some embodiments, the methods and uses described herein, such as the method of or use in treating absence seizures, such as absence seizures in childhood absence epilepsy (CAE) or juvenile absence epilepsy (JAE), in a human patient, reduces the number of seizures (e.g., daily) in the patient. In some embodiments, the administration (e.g., oral) of flunarizine to the patient provides a greater than 50%, greater than 55%, greater than 60%, greater than 65%, greater than 70%, greater than 75%, greater than 80%, greater than 85%, greater than 90%, or greater than 95% reduction in seizures. In some embodiments, the administration (e.g., oral) of flunarizine to the patient provides resolution of seizures (e.g., a 100% reduction in seizures). In some embodiments, the administration (e.g., oral) of flunarizine to the patient provides a reduction in seizures as shown in Table 2 and Table 3 in the examples section below.

In some embodiments, the present disclosure provides a method of reducing seizures in patient need thereof, comprising administering to the patient in need thereof a therapeutically effective amount of flunarizine. In some embodiments of the method of reducing seizures in patient need thereof, the patient is suffering from childhood absence epilepsy (CAE) or juvenile absence epilepsy (JAE). In some embodiments, the effective amount of flunarizine used in the methods of reducing seizures include the amounts of flunarizine described herein (e.g., 5 or 10 mg of flunarizine administered daily).

In certain embodiments, the methods and uses described herein comprise administering flunarizine in the form of a pharmaceutically acceptable composition that comprises flunarizine and one or more pharmaceutically acceptable carriers or excipients. The amount of flunarizine included in these compositions may correspond to one or more of the amounts described herein. In some embodiments, the compositions are a unit dose.

Examples of pharmaceutically acceptable compositions that comprise flunarizine include solid formulations (such as tablets, capsules, lozenges, dragées, granules, powders, wafers, multi-particulates, and films), liquid formulations (such as aqueous solutions, elixirs, tinctures, slurries, suspensions, and dispersions), and aerosolized formulations (such as mists and sprays). Preferably, pharmaceutically acceptable compositions that comprise flunarizine are formulated for oral administration, but other routes (e.g., parenteral, inhalation, rectal, nasal, buccal, or vaginal) are also contemplated. In one embodiment, a pharmaceutically acceptable oral composition of flunarizine includes a pediatric suspension or granulate. All above-noted amounts of flunarizine may be included in such formulations, e.g., a capsule comprising 1, 2, 5, 10, 15, 10, 25, 30, or 35 mg of flunarizine.

In another embodiment, kits are provided for oral administration of flunarizine for the treatment of absence seizures, such as absence seizures in childhood absence epilepsy (CAE) upon oral administration. Such kits comprise a plurality of oral dosage unit forms of flunarizine in combination with instructions for orally administering of flunarizine.

Additional embodiments and examples of the present disclosure are described herein. These embodiments and examples are illustrative and should not be construed as limiting the scope of the claimed invention.

### 5. EXAMPLES

Purpose: To investigate whether patients with absence seizures treated with flunarizine tolerate the study drug safely and whether such patients demonstrate a decrease in total number of absence seizures per week compared to a baseline period. Seizure frequency is documented via seizure diaries and side effects and safety are closely monitored. This study informs the feasibility and utility of conducting a larger clinical trial.

Objectives: The primary objective is to assess the efficacy, safety and tolerability of flunarizine compared to a baseline period on absence seizures in patients with treatment refractory generalized epilepsy taking a minimum of one anti-seizure medication (ASM). The primary outcome measure is median percent change in weekly or monthly absence seizure frequency in patients treated with flunarizine compared with the baseline period. Median percent change in weekly or monthly absence seizure frequency is measured by evaluating patient seizure diaries, at baseline and at the end of the treatment period. Severity/duration and frequency of associated adverse events/serious adverse events (AEs/SAEs), and clinically significant changes in vital signs including blood pressure, pulse or weight are also evaluated.

The secondary objectives are to evaluate EEG changes in patients with absence epilepsy; to evaluate changes in quality of life for patients; and to evaluate global impression change and clinical impression of change. The secondary outcome measures include median percent change in the number of absence seizures on EEG, and bursts of spike/wave lasting 2 or more seconds in patients treated with flunarizine compared with the baseline period; Clinical and Patient Global Impression of Change scores during the flunarizine treatment period; median percent change in weekly absence seizure frequency from baseline compared to end of study period; and median percent change in weekly absence seizure frequency from flunarizine treatment period.

The following questionnaires are administered in order to assess the effect of flunarizine on the quality of life of participants:
ADHD Rating Scale-IV
Quick Inventory of Depressive Symptomatology Self-Report
Generalized Anxiety Disorder—7 Items
Quality of Life in Childhood Epilepsy Questionnaire

These are performed to enable the investigator to effectively address any negative side effects mentioned in the literature. These questionnaires are administered by the research coordinator at the baseline visit, at Visit 4 and at Visit 5.

5.1.1 Study Design

Study Treatment: Study participants receive flunarizine 5 mg capsules.

Study Outline: The procedures set out in this study protocol are designed to ensure that the investigator and their team abide by the principles of the current ICH GCP guideline on the conduct, evaluation, and documentation of this study, as described in ICH Topic E6 guideline. ICH GCP is an international ethical and scientific quality standard for designing conducting recording and reporting studies that involve the participation of human subjects. Compliance with this standard provides public assurance that the rights, safety, and well-being of study subjects are protected, and that the clinical study data are credible.

The study also is carried out according to all applicable international and national regulatory requirements.

This is an open-label, single-centre study to evaluate the clinical efficacy, safety and tolerability of flunarizine administered as adjunctive treatment in participants diagnosed with treatment resistant absence epilepsy. The study includes 20 participants.

The study is open for screening until the 20^{th} patient is screened and deemed eligible during the baseline visit. If at any point, a patient is withdrawn early, the study re-opens for screening in order to fulfil the required 20 patients.

Assuming little or no spontaneous reduction in seizure frequency without flunarizine treatment, the number of subjects experiencing a reduction of 50% or more of seizure frequency required to get power (1-β) of 0.80 or better with an a level of p≤0.05 is 8 for flunarizine to be superior to no intervention.

The study design includes a screening visit, a baseline visit, three treatment phase visits and one follow-up visit. If the participant chooses to take part in the study, they sign an informed consent form (ICF) at the screening visit before any study related procedures take place.

Early Study Termination: The investigator or the subject or caregiver can terminate the study at any time for any reason. If this is necessary, active participants in the study are contacted, requested to stop or wean the study treatment, and scheduled for a visit with the investigator as soon as possible, in which the end of treatment procedures are performed (see section 6.4.2). The investigator is responsible for informing the local REB and Health Canada of the study termination according to ICH-GCP regulations.

End of Study: The study ends when the 20^{th} study participant has completed the follow-up visit. Participants are seen four weeks after discontinuation of flunarizine to assess any adverse events. Once the study is over, the data are locked and the report is written.

5.1.2 Population

Inclusion Criteria: Participants eligible for this study meet all of the following criteria:
1. Participant is 4 to 18 years of age
2. Participant has treatment resistant absence seizures. Treatment resistant absence seizures are defined as failure to respond to 9 or more appropriate anti-seizure medications (ASMs). The potential participant must have failed to respond to or tolerate appropriate doses of two of the following: ethosuximide (>/=20 mg/kg/day), valproic acid/divalproex sodium (>/=15 mg/kg/day) or lamotrigine, (>/=5 mg/kg/day).
3. Participant must currently be taking a minimum of one ASM
4. Normal development (may have specific learning disabilities and/or ADHD/ADD)
5. EEG confirmation of absence seizures and EEG within 2 months of screening visit
6. Absence seizures reported at least 10 times per week
7. Participants must have no changes in baseline ASMs for 1 month prior to screening period
8. Participant or caregiver can maintain a seizure diary
9. Participant is able to swallow capsules
10. Written informed consent must be provided. Legal guardians of the participants must be able to understand and provide written consent on behalf of the participant, since study participants may be too young to provide informed consent. For participants 7-18 years of age, assent is required along with written informed consent of the caregiver.
11. The participant is willing and able to attend all study visits
12. Participant has the ability to speak and read in English
13. Females of child bearing potential who are sexually active must be willing to use contraceptive methods for the duration of the study and 6 months post last dose of study drug.
14. Males who are sexually active with females of child bearing potential must be willing to use contraception for the duration of the study and 3 months post last dose of study drug.

Exclusion criteria: Participants eligible for this study do not meet any of the following criteria:
1. Age<4 years, >18 years
2. Participant has uncontrolled myoclonic or generalized tonic clonic seizures
3. Psychogenic non-epileptic seizures
4. Known diagnosis of intellectual disability
5. Autism spectrum disorder
6. Participant is taking carbamazepine, oxcarbazepine, phenytoin, eslicarbazepine, or vigabatrin
7. History of poor compliance with medication
8. Inability of parent or caregiver to maintain a seizure diary
9. History of depression or psychosis
10. Participant has previously taken flunarizine and has had an adverse reaction to treatment
11. Participants who are pregnant or breastfeeding
12. Presence of extrapyramidal symptoms
13. Participants who have clinically significant hepatic impairment as assessed by the investigator

5.1.3 Treatment

Study Treatment: Study participants receive flunarizine 5 mg capsules.

Starting Dose: The starting dose is one 5 mg capsule taken orally at night.

5.1.4 Drug Administration

The initial dose of flunarizine at Visit 2 is 5 mg/day taken at night. The dose is taken orally as one 5 mg capsule. In order to optimize response to treatment, dose escalation, at the discretion of the investigator, is performed at Visit 3. If the 5 mg/day dose is tolerated by any given participant, the dose is escalated to 10 mg/day taken as two 5 mg capsules. Escalation of the dose at Visit 3 does not occur if the investigator considers seizure control to be adequate or any adverse events to be dose limiting. The investigator uses discretion to maintain or adjust the dose level based on the study participant's tolerability and clinical response, alongside other medical considerations. If deemed necessary by the investigator, the 10 mg dose is be split into morning and evening doses to reduce minor side effects.

Concomitant Medication: The study participant is instructed not to take any medications (over-the-counter or other products) during the protocol treatment period without prior consultation with the investigator, unless considered emergent therapy, such as antibiotics or for resuscitation. The investigator instructs the study participant to inform them if any medication not previously recorded is taken during the study. All medications (other than study drug) taken when starting study treatment are reported on the Concomitant Medication CRF. The study participant is permitted to take ethosuximide, divalproex sodium, lamotrigine, perampanel, topiramate, clonazepam, acetazolamide and/or levetiracetam, but they must not be taking carbamazepine, oxcarbazepine, phenytoin, lacosamide, or vigabatrin.

Study Drug Dispensing: The investigator gives thorough instructions to the caregiver on how to administer the drug. The investigator instructs the participant to take the study drug exactly as prescribed. The study participant receives the drug from the investigator or designated study staff at the site. Each month the family should bring their study medication to the site and the investigator. All dosages for the drug are recorded in the Dosage Administration Record CRF. The drug is dispensed in bottles, prepared and dispensed by the pharmacy.

Drug Supply and Storage: Flunarizine commercial supply is shipped to BC Children's Hospital Pharmacy by AA Pharmaceuticals. Study medication must be received by designated personnel at the study site, handled and stored safely and properly, and kept in a secured location to which only the designated site personnel (research pharmacist) have access. The study treatment is stored according to the instructions specified on the drug labels and in the Product Monograph.

Study Drug Compliance: Compliance is assessed by the investigator at each study visit. Information is collected on how much medication the participant has used by checking the number of tablets remaining. Each week the family should bring their medication and empty bottles to the site for the investigator to monitor. The study participant is asked to return all empty study drug boxes, unused study drug, and packaging at the end of the study or at the time of study treatment discontinuation.

5.1.5 Visit Schedule and Assessments

Participants for the study are selected by the investigators of this study. They are selected from the hospital patient database, and their eligibility is determined based on their epilepsy history as well as the outlined inclusion criteria.

At the investigator's discretion, the physical and neurological exams are performed over telephone or through a remote videoing system such as Skype for Business, and vital signs can be obtained locally by the family's General Practitioner.

Screening Visit: Before any screening procedure takes place, the informed consent form (ICF), which outlines all study requirements and procedures, are be signed by the participating family. They are informed that their participation is voluntary, and they can choose to withdraw from the study at any time without any consequences to their medical care or other services to which they are entitled to. The ICF is given to the participant at least 24 hours before the screening visit, ensuring that they understand all aspects of the study, and any questions are answered by the primary investigator to their satisfaction.

At the screening visit, the seizure history is assessed using detailed parental interviews and chart review. The investigator takes the participant's medical history (including date of seizure onset date of epilepsy diagnosis). The participant's vital signs (temperature, blood pressure and heart rate), height and weight are recorded. The participant's eligibility is confirmed, including absence seizure frequency and prior treatment with ASMs. From the screening visit until the end of the study, the participant's existing ASM treatment regimen (of 1 or more ASMs) remain unchanged. The family is told to contact the investigator before taking any concomitant medications while they are on the study drug. A physical and neurological examination is performed by the investigators. If the participant meets the eligibility criteria they are instructed on how to keep a seizure diary.

Screening Failures: Participants who sign an informed consent form but fail to be eligible for any reason outlined in the protocol, are considered a screen failure. No further data are recorded for these participants, unless they experience a serious adverse event during the screening phase, in which case they are followed clinically until the adverse event has stabilized or passed.

Baseline Visit: The baseline visit occurs four weeks after the screening visit. A physical and neurological examination are performed by the investigator, and vital signs, height and weight are collected by the designated research coordinator. The participant's seizure activity is assessed from the seizure diaries. Adverse events and concomitant medications are also assessed at this visit.

If the participant is a female of childbearing potential, they complete a urine pregnancy test. Pregnancy is an exclusion criterion, and any participants who are pregnant are discontinued on the study medication.

The following questionnaires are completed: ADHD Rating Scale-IV, Quick Inventory of Depressive Symptomatology Self-Report, Generalized Anxiety Disorder—7 Items and the Quality of Life in Childhood Epilepsy Questionnaire. During the baseline visit a 1 month supply of the study drug, flunarizine, is dispensed and administration guidelines are provided.

Treatment Phase: The treatment phase begins at baseline visit and continues for 12 weeks. The study participant has three visits during the treatment period.

The first visit during treatment phase occurs 4 weeks after the baseline visit and the participant undergoes the following assessments:
Physical and neurological exams
Collection of vital signs
Evaluation of seizure diary

Adverse events and drug accountability are also assessed at during this visit. A four week supply of flunarizine is dispensed. The dose of flunarizine is increased depending on seizure control and adverse events by the investigator.

The second and third visits during treatment phase occur 8 and 12 weeks after the baseline visit respectively and the study participant undergoes the following assessments:
Physical and neurological exams
Collection of vital signs
Evaluation of seizure diary
Clinical global impression of change Participant global impression of change
EEG (only done during the second visit: week 8 of treatment)
An additional four week supply of flunarizine is dispensed at the second visit (week 8 of treatment).
Completion of the following questionnaires: ADHD Rating Scale-IV, Quick Inventory of Depressive Symptomatology Self-Report, Generalized Anxiety Disorder—7 Items and the Quality of Life in Childhood Epilepsy Questionnaire (performed by the caregiver in the presence of the clinical research coordinator)

Adverse events and drug accountability are also assessed during these visits.

End of Study: At the discretion of the investigator, at the End of Treatment period visit (Visit 5), flunarizine is prescribed “off label” to study participants, if the benefit of the study drug is apparent, and no adverse events are noted. This is in accordance to Health Canada Division 5 guidelines.

Premature Withdrawal: Participants withdraw from the study or are excluded at the investigator's discretion at any time. Participants are withdrawn from the study if any of the following occur:
Serious adverse events that are suspected to be related to study medication based on previous literature
History of poor compliance with medication
Inability to maintain a seizure diary
Withdrawal of consent
If subject becomes pregnant during the study duration

Participants who are withdrawn prematurely have an End of Treatment visit as close as possible to the day that the study drug is stopped. There is then a follow-up visit 30 days after study drug is stopped, during which time AEs and seizure frequencies continue to be recorded.

If premature withdrawal occurs for any reason, the investigator makes an effort to determine the primary reason for a subject's premature withdrawal and record it on the CRF. All subjects who prematurely withdraw from the study with an ongoing AE are be followed until the event is resolved or deemed stable. If a subject withdraws prematurely after dosing, all data to be collected prior to discharge are collected at the time of premature discontinuation.

Follow-up/Study Completion: Participants are followed for 30 days after study treatment ceases for adverse events, and are scheduled for a follow-up clinic visit at the end of this 30 day period. The investigator evaluates the seizure diary and ask the participant about adverse events.

Participants whose treatment is interrupted or permanently discontinued due to an adverse event are followed until resolution or stabilization of the event, whichever comes first. If participants are lost to follow-up, study staff record any attempted correspondence with them (phone calls, letters, etc.).

Participants continue using contraception 6 months post last dose of the study drug.

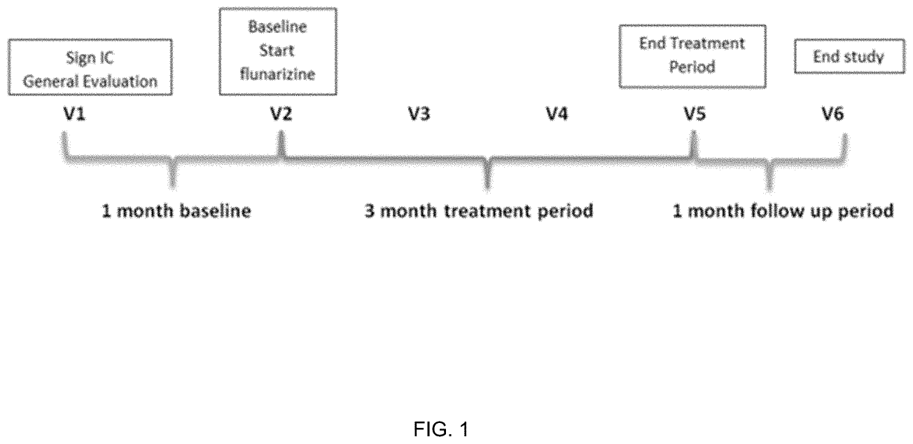

FIG. 1 provides a summary timeline of visits and treatments and Table 1 provides a summary of visits and assessments. The primary endpoint is the change in absence seizure frequency per 30 days between the baseline and the final 30-days of treatment.

TABLE 1
Summary of Visits and Assessments.

| | Screening | Baseline | Treatment | | | Follow-up |
|---|---|---|---|---|---|---|
| | Visit Number | | | | | |
| Study Period | 1 | 2 | 3 | 4 | 5 | 6 |
| Week | −4 | 0 | +4 | +8 | +12 | +16 |
| Informed consent | X | | | | | |
| Eligibility assessment | X | | | | | |
| Medical history | X | | | | | |
| Physical and neurologic examinations | X | X | X | X | X | |
| Vital signs | X | X | X | X | X | |
| Height, weight, body mass index | X | | X | X | X | |
| Concomitant medications registry | X | X | | | | |
| EEG | | | | X | | |
| Urine pregnancy test | | X | | | | |
| Adverse events | | X | X | X | X | X |
| Clinical global impression of change | | | | X | X | |
| Patient global impression of change | | | | X | X | |
| ADHD Rating Scale-IV Questionnaire | | X | | X | X | |
| Quick Inventory of Depressive | | X | | X | X | |
| Symptomatology Self-Report | | | | | | |
| Generalized Anxiety Disorder - 7 Items | | X | | X | X | |
| Questionnaire | | | | | | |
| Quality of Life in Childhood Epilepsy | | X | | X | X | |
| Questionnaire | | | | | | |
| Training and compliance check of Diary | X | X | X | X | X | X |
| Dispense study drug to participant | | X | X | X | | |
| Study drug compliance check | | | X | X | X | |

5.1.6 Preliminary Results

Initially, patients with Jeavons Syndrome, CAE (including those in whom CAE has persisted into adolescence), and JAE were enrolled. Table 2 shows preliminary demographics and study participant overview and Table 3 shows the number of absence seizures during EEG.

TABLE 2
Preliminary Demographics and Study Participant Overview

| | | Age at | | | | | Baseline | | | | |
|---|---|---|---|---|---|---|---|---|---|---|---|
| | | Seizure | Age at | # of | | | Seizure | Seizure | % | Daily Dose | |
| Patient/ | Epilepsy | Onset | Screening | Failed | Current | Ketogenic | Frequency | Frequency | Reduction | of | |
| Sex | Syndrome | (yrs) | (yrs) | ASMs | ASMs | Diet | per Month | at visit 5 | in Seizures | Flunarizine | |
| 1F | Jeavons | 5.5 | 16 | 7 | VPA | No | 409 | 608 | ~50% | 10 | mg |
| | | | | | CLON | | | | Increase | | |
| 2F | Jeavons | 6 | 14 | 6 | VPA | Yes | 334 | N/A | Early | 5 | mg |
| | | | | | | | | | Termination | | |
| 3F | CAE | 3 | 16 | 7 | LTG | Yes | 41 | 6 | 85% | 10 | mg |
| | | | | | ESM | | | | | | |
| 4F | CAE | 7 | 15 | 3 | LTG | No | 79 | 34 | 57% | 10 | mg |
| 5F | CAE | 2 | 5 | 2 | VPA | No | 54 | 9 | 83% | 5 | mg |
| 6F** | JAE | 9 | 13 | 3 | LTG | No | 90 | — | — | 10 | mg |

TABLE 3
Number of Absence Seizures during EEG

| | | | % Reduction in |
|---|---|---|---|
| Patent # | Baseline | Visit 5 | Seizures (EEG) |
| 1 | 9 | 8 | 11 |
| 2 | 5 | Early | N/A |
| | | Termination | |
| 3 | 16 | 1 | 94 |
| 4 | 7 | 0 | 100 |
| 5 | 5 | 0 | 100 |
| 6 | 6 | 3 | 50 to 100%*** |

**Subject 6. Family was very unreliable with keeping seizure diaries.
***Subject 6. During baseline EEG lasting 36 minutes she had 6 seizures, 4 during hyperventilation lasting 6 to 9 seconds and 2 spontaneous seizures lasting 9 and 10 seconds. On flunarizine 10 mgs during 36-minute EEG had no clinical absence seizures, there were 2 bursts of spike wave lasting 6 seconds with no clinical signs and one burst for 2 seconds.

Flunarizine resulted in >50% reduction in diary recorded seizures in all 3 CAE patients, with 2 snowing >80% reduction.

EEGs for the three CAE subjects showed resolution of absence seizures for 2 subjects and 94% reduction for one.

Two subjects had the onset of absence seizure in early childhood with persistence into teenage years, which demonstrates the spectrum of absence seizures across age groups, including those for JME and JAE.

EEG data for the single JAE subject showed a 50 to 100% reduction in seizures.

The two patients with Jeavons Syndrome did not respond to flunarizine. One had increased seizures and one terminated within one week due to increase in seizures.

This evidence suggests that flunarizine is effective for treatment of CAE and JAE, including CAE and JAE in patients experiencing persistent absence seizures and CAE and JAE patients having exhibited treatment resistance to one or more ASMs.

Flunarizine was well tolerated with weight gain as the only side effect in one patient resulting in medication discontinuation. Depressive symptoms and somnolence were not observed in any patient. No significant change was observed for ADHD and QOL questionnaires.

All of the U.S. patents, U.S. patent application publications, U.S. patent applications, foreign patents, foreign patent applications, and non-patent publications, including U.S. provisional appl. No. 63/116,881, filed Nov. 22, 2020, referred to in this specification are incorporated herein by reference in their entireties.

Although the foregoing compositions, methods, and uses have been described in some detail to facilitate understanding, it will be apparent that certain changes and modifications may be practiced within the scope of the appended claims. Accordingly, the described embodiments are to be considered as illustrative and not restrictive, and the claimed invention is not to be limited to the details given herein, but may be modified within the scope and equivalents of the appended claims.

1. A method for treating childhood absence epilepsy (CAE) or juvenile absence epilepsy (JAE) in a human patient, comprising administering to a patient in need thereof a therapeutically effective amount of flunarizine.

2. The method of claim 1, wherein the patient has exhibited treatment resistance to one or more epilepsy treatments.

3. The method of claim 1, wherein the flunarizine is administered to the patient as a first-line treatment.

4. A method for treating absence epilepsy or absence seizures in a human patient in need thereof, comprising administering a therapeutically effective amount of flunarizine to the patient;
wherein the patient has exhibited treatment resistance to one or more epilepsy treatments.

5. A method for treating absence epilepsy or absence seizures in a human patient in need thereof, comprising
identifying a patient that has exhibited treatment resistance to one or more epilepsy treatments; and
administering a therapeutically effective amount of flunarizine to the patient.

6. The method of claim 1, wherein the flunarizine is administered to the patient as a second-line or later treatment for the absence epilepsy or absence seizures.

7. The method of claim 1, wherein the patient exhibits CAE.

8. The method of claim 1, wherein the patient exhibits JAE.

9. The method of claim 1, wherein the patient does not exhibit Jeavons syndrome.

10. The method of claim 1 wherein the patient does not exhibit Lennox-Gastaut Syndrome.

11. The method of claim 1, wherein the patient is less than 21 years of age.

12. The method of claim 11, wherein the patient is from 3 to 20 years of age.

13. The method of claim 12 wherein the patient is from 4 to 18 years of age.

14. The method of claim 1, wherein the patient exhibits absence seizures with hyperventilation.

15. The method of claim 1 wherein the patient exhibits photosensitive epilepsy.

16. The method of claim 1 wherein the patient exhibits tonic-clonic seizures.

17. The method of claim 1, wherein the patient exhibits generalized polyspike or spike and wave discharges at 2 to 6 Hz when measured by electroencephalogram.

18. The method of claim 17, wherein the patient exhibits generalized spike and wave discharges at 3 Hz when measured by electroencephalogram.

19. The method of claim 1, wherein the patient has exhibited treatment resistance to two or more epilepsy treatments.

20. The method of claim 2, wherein the patient has exhibited treatment resistance to one or more of topiramate, zonisamide, levetiracetam, a benzodiazepine, stiripentol, amantadine, ethosuximide, valproic acid, valproate, divalproex sodium, or lamotrigine.

21. The method of claim 20 wherein the patient has exhibited treatment resistance to one or more of ethosuximide, valproic acid, valproate, or lamotrigine.

22. The method of claim 1, wherein the patient is concurrently being treated with one or more additional epilepsy treatments.

23. The method of claim 22, wherein the patient is concurrently being treated with two or more additional epilepsy treatments.

24. The method of claim 22 wherein the patient is concurrently being treated with topiramate, zonisamide, levetiracetam, a benzodiazepine stiripentol, amantadine, ethosuximide, valproic acid, valproate, divalproex sodium, or lamotrigine.

25. The method of any one of claims claim 24, wherein the patient is concurrently being treated with ethosuximide, valproic acid, valproate, or lamotrigine.

26. The method of claim 1, wherein the patient is not concurrently being treated with an additional epilepsy treatment.

27. The method of claim 1 wherein the patient is on a ketogenic diet.

28. (canceled)

29. The method of claim 1 wherein the patient is female.

30. The method of claim 1 wherein the patient is male.

31. The method of claim 1, wherein the patient does not exhibit myoclonic absences epilepsy.

32. The method of claim 1 wherein 0.5-50 mg/day of flunarizine is administered to the patient.

33. The method of claim 1, wherein 1-25 mg/day of flunarizine is administered to the patient.

34. The method of claim 33, wherein 2-15 mg/day of flunarizine is administered to the patient.

35. The method of claim 34 wherein 5 mg/day of flunarizine is administered to the patient.

36. The method of claim 34, wherein 10 mg/day of flunarizine is administered to the patient.
